(19) United States
(12) Patent Application Publication
(10) Pub. No.: US 2025/0099148 A1
(43) Pub. Date: Mar. 27, 2025

(54) Heat Resistant Bone Screw

(71) Applicant: Howmedica Osteonics Corp., Mahwah, NJ (US)

(72) Inventor: Mayur J. Thakore, Parsippany, NJ (US)

(21) Appl. No.: 18/829,411

(22) Filed: Sep. 10, 2024

Related U.S. Application Data
(60) Provisional application No. 63/539,689, filed on Sep. 21, 2023.

(51) Int. Cl. A61B 17/86

(52) U.S. Cl. CPC A61B 17/866; A61B 2017/8655

(57) ABSTRACT

The present disclosure is directed towards a fastener for implanting into a bone, the fastener comprising a metallic shaft and an outer layer. The outer layer is disposed on an outer surface of the shaft, the outer layer being configured to limit the effect of an electromagnetic field on the metallic shaft, wherein the outer layer includes a threaded portion along its length.

### CROSS-REFERENCE TO RELATED APPLICATIONS

This application claims the benefit of the filing date of U.S. Provisional Patent Application No. 63/539,689, filed Sep. 21, 2023, the disclosure of which is hereby incorporated herein by reference.

### BACKGROUND

Magnetic resonance imaging (MRI) examinations for clinical diagnostics are conducted under magnetic radiofrequency (RF) fields and gradient-induced heating. When patients with metallic implants such as bone screws are scanned during an MRI examination, the scan may cause such metallic implants to dramatically increase in temperature. For example, RF-induced heating is proportionate to the length of the metallic implant such that increasing implant length increases the probability of RF-induced heating to occur. Increases in the temperature of the metallic implants may cause a concomitant increase in temperature in bodily tissue in contact with or adjacent to the area where the metallic implant is disposed in the body.

These concerns with heat generation are exacerbated by the characteristics of the metallic implants typically used in surgical procedures. For example, the properties and dimensions of the implant, the magnetic properties and conductivity of the implant, the need for orientation of the implant in a particular manner in relation to the direction of the main magnetic field, the strength of the magnetic field, the pulse sequence type, and the impact of other MRI examination imaging parameters. Additionally, metallic implants have a high potential for heating along sharp edges, points, or pointed loops, characteristics that may be present in bone screws, for example, which may cause heating of bodily tissue in contact with or adjacent to the area where the metallic implant is disposed.

Accordingly, a need exists to develop metallic implant designs less susceptible to heating when subject to scans such as MRI scans.

### BRIEF SUMMARY

In a first aspect, the present disclosure relates to a fastener for implanting into a bone. In a first example of a first embodiment, the fastener comprises a metallic shaft and an outer layer disposed on an outer surface of the shaft, the outer layer being configured to limit the effect of an electromagnetic field on the metallic shaft, wherein the outer layer includes a threaded portion along its length.

In a second example, the first example of the first embodiment may further be defined wherein the outer layer is a ceramic or polyether ether ketone (PEEK) material. In a third example, the first example of the first embodiment may further be defined wherein the outer layer is a coating and has a minimum thickness in a range from about 0.006 inches to about 0.030 inches.

In a fourth example, the first example of the first embodiment may further be defined wherein the outer layer is a cannulated sleeve that is removably attachable to the metallic shaft such that the cannulated sleeve encloses at least a majority of the metallic shaft. In a fifth example, the fourth example of the first embodiment may further be defined wherein the cannulated sleeve is open at a first end of the sleeve and at a second end of the sleeve opposite the first end such that a tip of the metallic shaft is exposed through one of the first and second ends of the sleeve. In a sixth example, the fourth example of the first embodiment may further be defined wherein the metallic shaft and the cannulated sleeve both include a plurality of flat surfaces meeting at sharp edges such that when the cannulated sleeve is disposed over the metallic shaft, the cannulated sleeve is prevented from rotating relative to the metallic shaft.

In a seventh example, the first example of the first embodiment may further be defined wherein the fastener is a bone screw and the metallic shaft includes a head at a proximal end of the metallic shaft. In an eighth example, the seventh example of the first embodiment may further be defined wherein the threaded portion extends to a distal tip of the fastener. In a ninth example, the first example of the first embodiment may further be defined wherein the outer layer is further configured to prevent radiofrequency exposure to the fastener from heating the metallic shaft.

In a first example of a second embodiment, a fastener may include a metallic shaft and an outer layer disposed on a surface of the metallic shaft, the outer layer being configured to prevent radiofrequency exposure to the fastener from heating the metallic shaft. In the first example, the outer layer may enclose a majority of the metallic shaft.

In a second example, the first example of the second embodiment may further be defined wherein a proximal end of the metallic shaft and a distal end of the metallic shaft define respective parts of an outer surface of the fastener and a remaining portion of the metallic shaft therebetween is enclosed by the outer layer. In a third example, the second example of the second embodiment may further be defined wherein the fastener is a bone screw and the proximal end of the metallic shaft is a head of the bone screw. In a fourth example, the third example of the second embodiment may further be defined wherein the distal end of the metallic shaft is a distal tip of the bone screw.

In a fifth example, the first example of the second embodiment may further be defined wherein the outer layer is a ceramic or polyether ether ketone (PEEK) material. In a sixth example, the first example of the second embodiment may further be defined wherein the outer layer is a coating and has a minimum thickness in a range from about 0.006 inches to about 0.030 inches.

In a seventh example, the first example of the second embodiment may further be defined wherein the outer layer is a cannulated sleeve that is removably attachable to the metallic shaft. In an eighth example, the seventh example of the second embodiment may further be defined wherein the cannulated sleeve is open at a first end of the sleeve and at a second end of the sleeve opposite the first end such that a tip of the metallic shaft is exposed through one of the first and second ends of the sleeve. In a ninth example, the seventh example of the second embodiment may further be defined wherein the metallic shaft and the cannulated sleeve both include a plurality of flat surfaces meeting at sharp edges such that when the cannulated sleeve is disposed over the metallic shaft, the cannulated sleeve is prevented from rotating relative to the metallic shaft. In a tenth example, the first example of the second embodiment may further be defined wherein the outer layer is further configured to limit the effect of an electromagnetic field on the metallic shaft.

In a second aspect, the present disclose relates to a method of using an implant in a surgical procedure. In a first example of a first embodiment of such method, the method includes the following steps: anchoring a bone screw in a bone of a patient or in a prosthesis disposed in the patient, the bone screw including a metallic shaft and a ceramic or polyether ether ketone (PEEK) outer layer enclosing an outer surface of the metallic shaft; and conducting a magnetic resonance imaging (MRI) scan of the patient, wherein the ceramic or PEEK coating reduces temperature changes in the metallic shaft due to the scan that would occur without the ceramic or PEEK coating.

### BRIEF DESCRIPTION OF THE DRAWINGS

FIG. 1A is a perspective view of a bone screw according to an embodiment of the present disclosure.

FIG. 1B. is a cross-sectional view of the bone screw of FIG. 1A.

FIG. 1C is an exploded view of the bone screw of FIG. 1A.

FIG. 1D is a close-up perspective view of a tip of the bone screw of FIG. 1A.

FIG. 2A is a perspective view of a bone screw according to another embodiment of the present disclosure.

FIG. 2B is a close-up partial cross-sectional view of the bone screw of FIG. 2A.

FIG. 3A is a perspective view of a bone screw according to another embodiment of the present disclosure.

FIG. 3B is a close-up partial cross-sectional view of the bone screw of FIG. 3A.

FIG. 4A is a perspective view of a bone screw according to another embodiment of the present disclosure.

FIG. 4B is a close-up partial cross-sectional view of the bone screw of FIG. 4A.

FIG. 5A is a perspective view of a bone screw according to another embodiment of the present disclosure.

FIG. 5B is a close-up partial cross-sectional view of the bone screw of FIG. 5A.

FIG. 6A is a perspective view of an ASTM phantom and RF coils according to a simulated experiment setup of an embodiment of the present disclosure.

FIG. 6B is perspective view of a simulated knee replacement construct positioned within the ASTM phantom and RF coils of the simulated experimental setup of FIG. 6A.

### DETAILED DESCRIPTION

As used herein, the terms “about,” “generally,” and “substantially” are intended to mean that slight deviations from absolute are included within the scope of the term so modified. To aid the Patent Office and any readers of any patent issued on this application in interpreting the claims appended hereto, Applicant notes that it does not intend any of the appended claims or claim elements to invoke 35 U.S.C. § 112 (f) unless the words “means for” or “step for” are explicitly used in the particular claim.

As used herein, the term “proximal,” when used in connection with a surgical tool or device, or components of a device, refers to the end of the device closer to the user of the device when the device is being used as intended. On the other hand, the term “distal,” when used in connection with a surgical tool or device, or components of a device, refers to the end of the device farther away from the user when the device is being used as intended.

In one aspect, the present disclosure relates to implants used in surgical procedures. In specific embodiments described herein, such implants may be bone screws. However, it should be appreciated that the present disclosure is not limited to bone screws and the principles of the present disclosure may be applied to other implants. For example, other metallic implants.

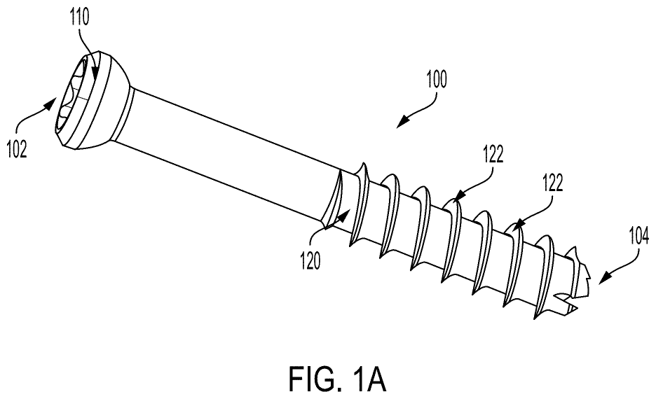

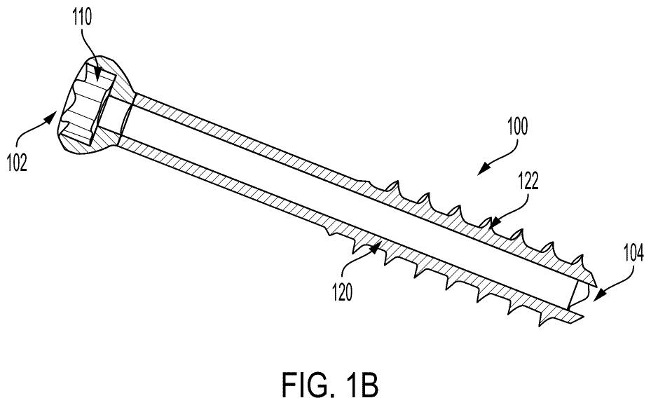

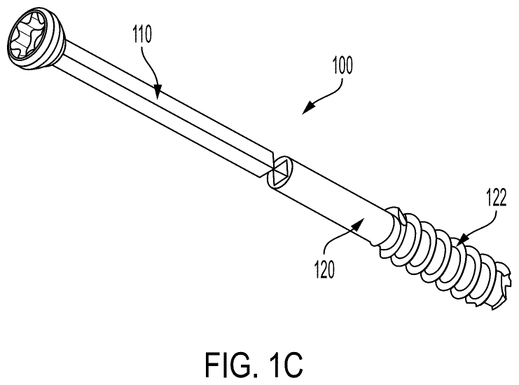

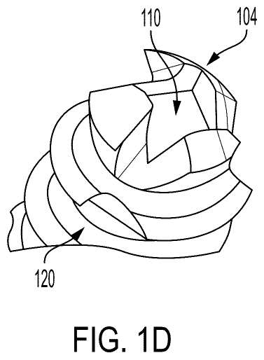

According to one embodiment of the present disclosure, a metallic implant in the form of a bone screw 100 is shown in FIGS. 1A-1D. Bone screw 100 includes a drive or shaft 110 and a sleeve 120 disposed over shaft 110. Sleeve 120 may be attached to shaft 110 by any mechanical adhesive process. Shaft 110 is metallic and in some examples may be titanium, a titanium alloy, stainless steel, a stainless steel alloy, cobalt-chrome, cobalt alloys, nickel titanium, or nickel titanium alloys. Sleeve 120 is a ceramic and/or polyether ether ketone (PEEK) material. Further details on the characteristics of the ceramic or PEEK nature of sleeve 120 are provided below. Bone screw 100 may be implanted into a bone of a patient or may serve another function in conjunction with other implants utilized in a surgical procedure.

As depicted, shaft 110 includes a head 102 with a drive for actuation of the screw and a body portion in the form of a rectangular prism, as shown in FIG. 1C. It should be appreciated that the shape of the shaft 110 body may vary from that shown. For example, the body may have three sides or five sides, among other characteristics. Sleeve 120 is cannulated and has an interior surface to complement the shaft 110 body. In this manner, the sleeve 120 is slidable over the body of the shaft 119. When combined in such manner, rotation of sleeve 120 relative to shaft 110 is prevented due to the edges of the shaft 110 body, such as the rectangular prism shape of the shaft 110 shown in FIG. 1C. The outer surface of sleeve 120 may be threaded 122 as shown in FIGS. 1A-1D. The threaded portion 122 may extend to a distal tip 104 of the sleeve 120, as shown in FIG. 1D. In some examples, and as shown in FIG. 1D, the cannulation of sleeve 120 may extend entirely through the sleeve 120. One purpose of sleeve 120 of bone screw 100 is to prevent or substantially limit the extent to which bone screw 100 increases in temperature, i.e., heats up, under radiofrequency (RF) exposure. Through such screw characteristics, MRI scans of such screws will be much less likely to cause an increase in the temperature of bodily tissue and organs proximate to the bone screw during the scanning procedure.

Sleeve 120 includes a unique design, geometry, and dimensions to eliminate sensitive surfaces. Sleeve 120 blocks the eddy current effects between the implant and RF-field exposure. In addition to this, sleeve 120 provides a reduction in the MRI examination artifacts generated by bone screw 120 and other metallic implants. Such sleeve 120 characteristics also apply for sleeves 120 designed for other metallic implant types.

Sleeve 120 is comprised of any grade of ceramic or PEEK material. In one embodiment, shaft 120 may be comprised of a hydroxyapatite bioactive ceramic or PEEK. The ceramic or PEEK material and threaded portion 122 of sleeve 120 enhance fixation within the bone and bodily tissue, which in turn will increase grounding within the bone and bodily tissue. The ceramic or PEEK material of sleeve 120 also provides improved insulation of shaft 120 and more evenly distributes thermo-dynamic effects relative to a purely metallic implant. As such, sleeve 120 has a low dielectric loss allowing for the significantly reduced dissipation of energy in high-level electromagnetic fields. Sleeve 120 may have a high transparency. Such transparency reduces the free electrons that cause diagnostics artifacts and thermal instability. Additionally, sleeve 120 allows the energy induced during RF exposure or MRI examinations to be disseminated through a proximal end 102 or head of bone screw 100 or through a distal end 104 or tip of bone screw 100.

A thickness of sleeve 120 for bone screw 100 may be determined based on the ability of the sleeve 120 to withstand forces expected during wear post-surgery, including any tension and/or compression. The thickness of the sleeve 120 may be uniform throughout, vary along different portions of the bone screw, and/or may vary along individual threads 122 of the sleeve.

Bone screw 100 may be manufactured using subtractive processes such as machining or through additive layer manufacturing, such as Shape Deposition Manufacturing (“SDM”), Selective Laser Power Processing (“SLPP”), Direct Metal Laser Sintering (“DMLS”), Selective Laser Sintering (“SLS’), Selective Laser Melting (“SLM”), Selecting Heating Sintering (“SHS”), Electron Beam Melting (“EBM”), material jetting, binder jetting, or the like. Additive layer manufacturing may be performed as described in U.S. Pat. Nos. 8,590,157, 11,006,981, and 11,701,146, the entire contents of which are hereby incorporated by reference herein.

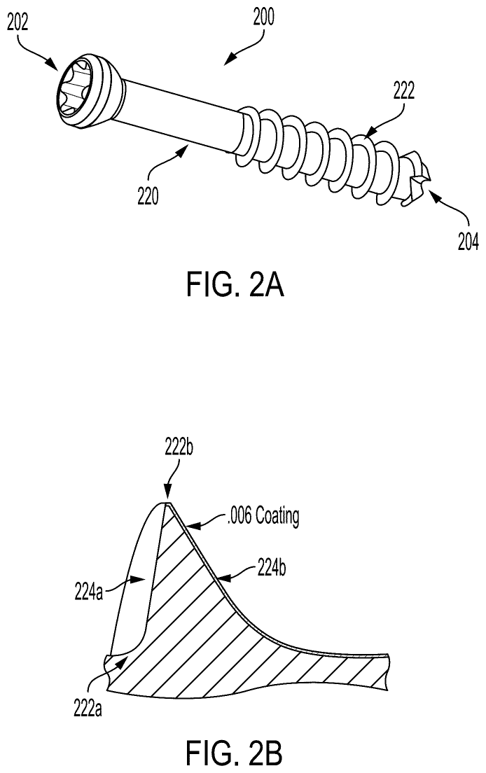

In other embodiments, a bone screw may include a metallic shaft with a ceramic or PEEK coating. FIGS. 2A-5B illustrate examples of such bone screw embodiments via bone screws 200, 300, 400, 500 having various thicknesses of coating 220, 320, 420, 520 on the respective bone screw. The thickness of coating 220, 320, 420, 520 improves fixation and reduces MRI examination artifacts.

A thickness of the coating in bone screws 200, 300, 400, 500 may be in a range from about 0.006 inches to about 0.030 inches. The range of coating thicknesses provides an optimized ceramic or PEEK coating in that the upper limit avoids larger thicknesses that may result in the coating peeling off the metallic body and that may result in the deposit of additional residues in the body, while the lower limit avoids thinner coatings that may not sustain insulative properties in the long run due to normal wear in the implant region of the patient. For example, a thin coating may deteriorate due to a rubbing effect that may occur with use of the patient's joint.

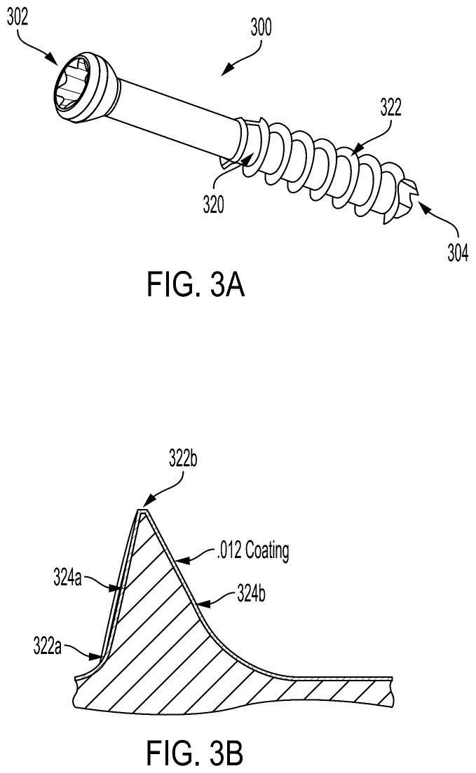

FIGS. 2A-5B illustrate specific examples of bone screws with ceramic or PEEK coating. The thickness of coatings 220, 320, 420, 520 ensures adhesion to the screw shaft surface and that the bone screw possesses insulative endurance. In FIGS. 2A-2B, bone screw 200 is shown with coating 220 having a thickness of about 0.006 inches on a surface of the screw shaft. In FIG. 2B, the part of the thread shown includes a root 222a, a crest 222b and flanks 224a, 224b between the root and the crest. In FIG. 2B, a portion of the screw thread is visible into the page on the left side of the cross-section, and for this reason, the coating appears different on flank 224a compared to flank 224b. However, the coating has a similar thickness on both flanks 224a, 224b. A similar visual effect is present on screws 300, 400, 500 described below and shown in FIGS. 3B, 4B and 5B. Returning to screw 200, the thickness of coating 220 is uniform along a length of the screw 200 but may vary slightly in certain surface regions. Excess coating thickness and/or variations in coating thickness may be present on parts of the screw surface, for example, based on the use of certain coating techniques.

In FIGS. 3A-3B bone screw 300 is shown with coating 320 thickness of about 0.012 inches on a surface of the screw shaft. In FIG. 3B, the part of the thread shown includes a root 322a, a crest 322b and flanks 324a, 324b between the root and the crest. The thickness of coating 320 is uniform along a length of the screw but may vary in certain regions. Excess coating thickness and/or variations in coating thickness may be present on parts of the screw surface, for example, based on the use of certain coating techniques.

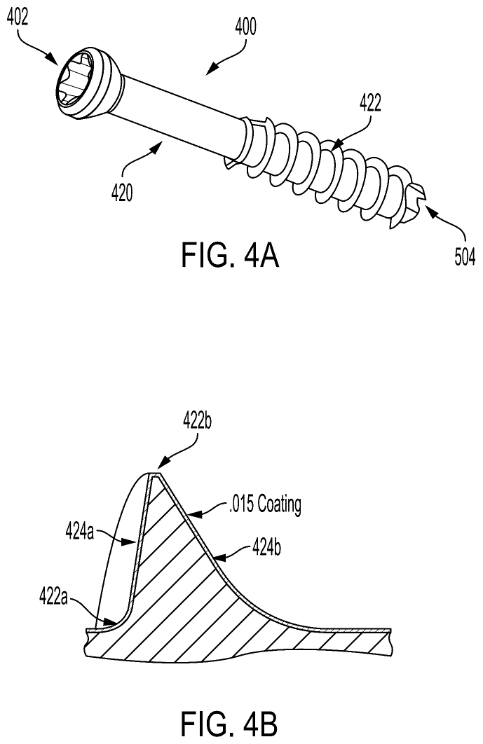

In FIGS. 4A-4B, bone screw 400 is shown with coating 420 thickness of about 0.015 inches on a surface of the screw shaft. In FIG. 4B, the part of the thread shown includes a root 422a, a crest 422b and flanks 424a, 424b between the root and the crest. The thickness of coating 420 is uniform along a length of the screw but may vary in certain regions. Excess coating thickness and/or variations in coating thickness may be present on parts of the screw surface, for example, based on the use of certain coating techniques.

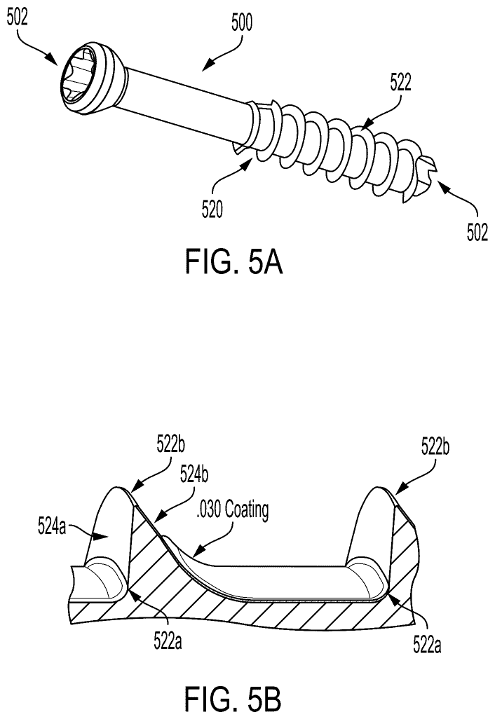

In FIGS. 5A-5B, bone screw 500 is shown with coating 520 thickness of about 0.030 inches on a surface of the screw shaft. In FIG. 5B, the part of the thread shown includes a root 522a, a crest 522b and flanks 524a, 524b between the root and the crest. The thickness of coating 520 is uniform along a length of the screw but may vary in certain regions. Excess coating thickness and/or variations in coating thickness may be present on parts of the screw surface, for example, based on the use of certain coating techniques.

Coatings 220, 320, 420, 520 may be fabricated using various methods such as physical vapor deposition, chemical vapor deposition (CVD), pulsed laser deposition (PLD), sputtering, or any other conventional bone screw generation with a secondary coating or adhesion.

In another aspect, the present disclosure relates to a method of using an implant as contemplated in the various embodiments described herein. For example, bone screw 100, 200, 300, 400, 500 may be implanted in a patient according to method steps set forth for a particular surgical procedure. As part of the procedure, MRI scans may be taken without causing a heating effect on the bone screws or at least significantly minimizing a heating effect on the bone screws 100, 200, 300, 400, 500 when they are present within a body of the patient. Further, because the bone screws 100, 200, 300, 400, 500 do not substantively increase in temperature, tissue in surrounding regions of the body similarly does not undergo temperature increases.

### Example

Hereinafter, the present disclosure will be described according to a specific example of a simulated experiment performed. During the experiment, simulated bone screws corresponding to bone screws 100, 200, 300, 400, 500 were exposed to computer simulations of a 1.5 Tesla (T) magnetic resonance (MR) environment for evaluating the effects of the respective sleeve 120 or coatings 220, 320, 420, 520.

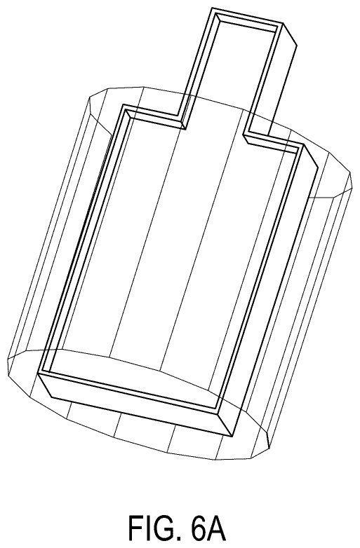

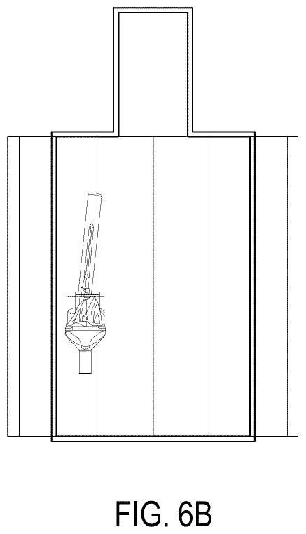

A Sim4Life™ software package (version 7.2), co-developed by IT′IS and ZMT Zurich MedTech AG, was used to evaluate heating patterns for simulated bone screws placed inside the gel of an ASTM phantom (ASTM F2182-19e2) under 1.5 T or 3.0 T MRI systems. The RF body coils at 1.5 T and 3.0 T were modeled using sixteen rungs with two end rings as shown in FIG. 6A to generate desired circular polarized B1+ fields, respectively. Here, the body of the ASTM phantom was placed at the center of the RF coil.

As an initial step, the electric field distribution pattern inside the ASTM phantom was investigated with and without the bone to understand the effect of bone on incident field variation. During each simulation of the experiment, medical devices, including a simulated bone screw, including some similar to that of at least one of the bone screws 100, 200, 300, 400, 500, were placed at an isocenter and 2 cm from the sidewall of the ASTM phantom. For the 1.5 T system, incident field strength was approximately 115 v/m (RMS value). Similarly, for the 3 T system, the incident field strength was approximately 100 v/m (RMS value). In both systems, the simulated whole-body MRI specific absorption rate (SAR) was at 2 W/kg, which corresponds to a normal operation mode in MRI applications. In addition, the excitation sources are the quadrature (circular polarized) mode. For each simulation of the experiment, electromagnetic simulations were executed to compute the RF-induced heating of the simulated bone screws with and without non-conductive coating or insulation layer. While simulations were run only of the simulated bone screws, an illustration of an example of a simulated knee replacement construct including, a simulated bone screw, and its loading position inside the ASTM phantom is shown in FIG. 6B. As illustrated, the center point of the ASTM phantom trunk is at the isocenter of the RF coil.

Electromagnetic modeling of the simulated bone screws and ASTM phantom were generated based upon the list of material properties, as shown in Table 1 as follows:

TABLE 1

| Material | | Gel | | | | | Stainless | Ceramic |
|---|---|---|---|---|---|---|---|---|
| Property | Air | Phantom | Bone | CoCrMo | Ti6Al4V | PEEK | Steel | (Al2O3) |
| Electrical | 0 | 0.47 | 0.060/0.067 | 1.10E+06 | 5.90E+05 | 0 | 1.25E+06 | 0 |
| Conductivity | | | | | | | | |
| (S/m) | | | | | | | | |
| Relative | 1 | 80 | 16.7/14.7 | 1 | 1 | 3.2 | 1 | 9.6 |
| electric | | | | | | | | |
| permittivity | | | | | | | | |
| Relative | 1 | 1 | 1 | 1 | 1 | 1 | 1 | 1 |
| Magnetic | | | | | | | | |
| Permeability | | | | | | | | |
| Thermal | — | 0.6 | 0.32 | 13 | 7.2 | / | 15 | 25 |
| Conductivity | | | | | | | | |
| (W/m · K) | | | | | | | | |
| Density | — | 1000 | 1908 | 8300 | 4400 | / | 7900 | 4360 |
| (kg/m3) | | | | | | | | |
| Specific Heat | — | 4200 | 1312 | 450 | 550 | 1800 | 500 | 880 |
| (J/kg · K) | | | | | | | | |

Additionally, for the modeling, adaptive mesh size was used in all simulations. For the simulated medical device, the mesh size of 0.5/0.2 mm was used for all parts in all three directions of freedom (x, y, z). An adaptive mesh ratio of less than 1.2 was used away from the simulated medical device with a maximum mesh size of 5 mm inside the simulated gel. The simulated shell was meshed using a grid size of 5 mm. No assumption was made in the electromagnetic modeling to simplify the modeling geometries. Accurate CAD models were loaded into the phantom for electromagnetic modeling. Further, the perfect matched layer (PML) absorbing boundary condition was used in the modeling. To minimize the reflection, the strength of “High” was used. This corresponds to a reflection coefficient less than 1%.

As described above, each simulated device was placed 2 cm from the left wall of the phantom in the width direction (x-axis), in the center of height (z-axis), and oriented lengthwise in the bore direction (y-axis) direction as illustrated by the simulated example knee replacement construct in FIG. 6B. This positioning corresponds to a location with a very high electric incident field. Further, for each simulated experiment, it was assumed that the maximum device length along the MRI bore direction should generate the worst-case heating, as established in Yan Liu, Ji Chen, Frank G. Shellock, and Wolfgang Kainz “Computational and experimental studies of an orthopedic implant: MRI-related heating at 1.5 T/64-MHz and 3 T/128-MHz”, Journal of Magnetic Resonance Imaging 37, Article first published online: 31 Jul. 2012| DOI: 10.1002/jmri.23764. Additionally, it was assumed that orientations of smaller devices should not make much impact on the RF-induced heating. (Id.)

Further, for each simulation performed, a maximum of 1 g averaged SAR was extracted near the simulated medical device (this corresponds to the maximum temperature rise per FDA guidance). For each of the following experiments, the results of the SAR and temperature rises were normalized to the whole body (WB) averaged SAR of 2 W/kg, which corresponds to the maximum allowed power under the normal operating mode for MRI.

### Experiment 1

In Experiment 1, simulated bone screws comprising shafts of non-conductive materials, such as PEEK and ceramics, were tested. Each of these simulated bone screws included a metallic distal tip. Each of simulated bone screws #1-6 were simulated under RF-induced heating under a 1.5 T MR environment to determine SAR values. Further, simulated bone screws #1-3 were evaluated without being positioned within a simulated bone, while simulated bone screws #4-6 were evaluated while being positioned within a simulated bone. Table 2 below summarizes the results of Experiment 1. As the results show, SAR values were significantly lower for simulated bone screws with PEEK and ceramic shafts, indicative of a significantly attenuated heating effect relative to metallic screws.

TABLE 2

| | | | SAR |
|---|---|---|---|
| Simulated | | | [W/kg] |
| bone screw | Configuration | | 1.5 T |
| #1 | 5572-4540G (Total Length=1.704 in, Shaft | without | 54.8 |
| | Diameter = ~0.116 in, Thread Outer | bone | |
| | Diameter = 0.177 in) Fully Metallic Screw | | |
| #2 | 5572-4540G (P/M) (Total Length = 1.861 in, | | 9.4 |
| | Shaft Diameter = ~0.116 in, Thread Outer | | |
| | Diameter = 0.177 in) | | |
| | PEEK shaft and Metallic Sharp Tip | | |
| #3 | 5572-4540G (P/M) (Total Length = 1.861 in, | | 9.4 |
| | Shaft Diameter = ~0.116 in, Thread Outer | | |
| | Diameter = 0.177 in) | | |
| | Ceramic Shaft and Metallic Sharp Tip | | |
| #4 | 5572-4540G (Total Length = 1.704 in, Shaft | with bone | 39.0 |
| | Diameter = ~0.116 in, Thread Outer | | |
| | Diameter = 0.177 in) | | |
| | Fully Metallic Screw | | |
| #5 | 5572-4540G (P/M) (Total Length = 1.861 in, | | 9.6 |
| | Shaft Diameter = ~0.116 in, Thread Outer | | |
| | Diameter = 0.177 in) | | |
| | PEEK Shaft and Metallic Sharp Tip | | |
| #6 | 5572-4540G (P/M) (Total Length = 1.861 in, | | 9.6 |
| | Shaft Diameter = ~0.116 in, Thread Outer | | |
| | Diameter = 0.177 in) | | |
| | Ceramic Shaft and Metallic Sharp Tip | | |

### Experiment 2

In Experiment 2, simulated bone screws comprised a metallic inner core. Each of these simulated bone screws were insulated by a layer of non-conductive materials, such as PEEK and ceramics, along their respective shafts. However, the heads and distal tips of the simulated bone screws were not fully insulated by these materials. Similar to Experiment 1, each of simulated bone screws #1-6 were simulated under RF-induced heating under a 1.5 T MR environment to determine SAR values. Further, simulated bone screws #1-3 were evaluated without being positioned within a simulated bone, while simulated bone screws #4-6 were evaluated while being positioned within a simulated bone. Table 3 below summarizes the results of Experiment 2. As the results show, SAR values were significantly lower for simulated bone screws with PEEK and ceramic layers for insulating metallic cores, indicative of a significantly attenuated heating effect relative to metallic screws.

TABLE 3

| | | | SAR |
|---|---|---|---|
| Simulated | | | [W/kg] |
| bone screw | Configuration | | 1.5 T |
| #1 | DR193199 (Total Length = 0.823744 in, Shaft | without | 17.6 |
| | Diameter = 0.0826772 in, Thread Outer | bone | |
| | Diameter = 0.122835 in) | | |
| | Metallic screw | | |
| #2 | DR193199 (Total Length = 0.823744 in, Shaft | | 11.6 |
| | Diameter = 0.0826772 in, Thread Outer | | |
| | Diameter = 0.122835 in) | | |
| | Metallic square drive core with flat tip and | | |
| | PEEK insulation layer | | |
| #3 | DR193199 (Total Length = 0.823744 in, Shaft | | 11.4 |
| | Diameter = 0.0826772 in, Thread Outer | | |
| | Diameter = 0.122835 in) | | |
| | Metallic square drive core with flat tip and | | |
| | Ceramic insulation layer | | |
| #4 | DR193199 (Total Length = 0.823744 in, Shaft | | 13.3 |
| | Diameter = 0.0826772 in, Thread Outer | | |
| | Diameter = 0.122835 in) | | |
| | Metallic screw | | |
| | DR193199 (Total Length = 0.823744 in, Shaft | | |
| #5 | Diameter = 0.0826772 in, Thread Outer | | 9.3 |
| | Diameter = 0.122835 in) with | | |
| | Metallic square drive core and PEEK insulation bone | | |
| | layer | | |
| | DR193199 (Total Length = 0.823744 in, Shaft | | |
| #6 | Diameter = 0.0826772 in, Thread Outer | | 9.3 |
| | Diameter = 0.122835 in) | | |
| | Metallic square drive core with flat tip and | | |
| | Ceramic insulation layer | | |

### Experiment 3

In Experiment 3, different thicknesses of PEEK coating were evaluated for each respective simulated bone screw. Specifically, simulated bone screw #2 comprises a PEEK coating of 0.006 inches (similar to that of the coating 220 of bone screw 200), simulated bone screw #3 comprises a PEEK coating of 0.012 inches (similar to that of the coating 320 of bone screw 300), simulated bone screw #4 comprises a PEEK coating of 0.015 inches (similar to that of the coating 420 of bone screw 400), and simulated bone screw #5 comprises a PEEK coating of 0.030 inches (similar to that of the coating 520 of bone screw 500). Similar to Experiments 1 and 2, each of simulated bone screws #1-6 were simulated under RF-induced heating under a 1.5 T MR environment to determine SAR values. Table 4 below summarizes the results of Experiment 3. As the results show, SAR values decrease as the thickness of the PEEK or ceramic layer or shaft increases for the simulated bone screws, indicative of a significantly attenuated heating effect relative to metallic screws.

TABLE 4

| | | | SAR |
|---|---|---|---|
| Simulated | | | [W/kg] |
| bone screw | Configuration | | 1.5 T |
| #1 | DR192418 (Total Length = 0.746142 in, | without | 16.2 |
| | Uncoated Shaft Diameter = 0.0826772 in, | bone | |
| | Uncoated Thread Outer Diameter = 0.122835 | | |
| | in) | | |
| | Metallic Screw, Uncoated | | |
| | DR192418 (Total Length = 0.746142 in, | | |
| #2 | Uncoated Shaft Diameter = 0.0826772 in, | | 15.1 |
| | Uncoated Thread Outer Diameter = 0.122835 | | |
| | in) | | |
| | Metallic Screw with 0.006 in PEEK Coating | | |
| | DR192418 (Total Length = 0.746142 in, | | |
| | Uncoated Shaft Diameter = 0.0826772 in, | | |
| #3 | without | | 15.2 |
| | Uncoated Thread Outer Diameter = 0.122835 | | |
| | bone | | |
| | in) | | |
| | Metallic Screw with 0.012 in PEEK Coating | | |
| #4 | DR192418 (Total Length = 0.746142 in, | | 15.2 |
| | Uncoated Shaft Diameter = 0.0826772 in, | | |
| | Uncoated Thread Outer Diameter = 0.122835 | | |
| | in) | | |
| | Metallic Screw with 0.015 in PEEK Coating | | |
| #5 | DR192418 (Total Length = 0.746142 in, | | 9.3 |
| | Uncoated Shaft Diameter = 0.0826772 in, | | |
| | Uncoated thread Outer Diameter = 0.122835 | | |
| | in) | | |
| | Metallic Screw with 0.030 in PEEK Coating | | |

Although the invention herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A fastener for implanting into a bone, the fastener comprising:
a metallic shaft; and
an outer layer disposed on an outer surface of the shaft, the outer layer being configured to limit the effect of an electromagnetic field on the metallic shaft,
wherein the outer layer includes a threaded portion along its length.

2. The fastener of claim 1, wherein the outer layer is a ceramic or polyether ether ketone (PEEK) material.

3. The fastener of claim 1, wherein the outer layer is a coating and has a minimum thickness in a range from about 0.006 inches to about 0.030 inches.

4. The fastener of claim 1, wherein the outer layer is a cannulated sleeve that is removably attachable to the metallic shaft such that the cannulated sleeve encloses at least a majority of the metallic shaft.

5. The fastener of claim 4, wherein the cannulated sleeve is open at a first end of the sleeve and at a second end of the sleeve opposite the first end such that a tip of the metallic shaft is exposed through one of the first and second ends of the sleeve.

6. The fastener of claim 4, wherein the metallic shaft and the cannulated sleeve both include a plurality of flat surfaces meeting at sharp edges such that when the cannulated sleeve is disposed over the metallic shaft, the cannulated sleeve is prevented from rotating relative to the metallic shaft.

7. The fastener of claim 1, wherein the fastener is a bone screw and the metallic shaft includes a head at a proximal end of the metallic shaft.

8. The fastener of claim 7, wherein the threaded portion extends to a distal tip of the fastener.

9. The fastener of claim 1, wherein the outer layer is further configured to prevent radiofrequency exposure to the fastener from heating the metallic shaft.

10. A fastener for implanting into a bone, comprising:
a metallic shaft; and
an outer layer disposed on a surface of the metallic shaft, the outer layer being configured to prevent radiofrequency exposure to the fastener from heating the metallic shaft,
wherein the outer layer encloses a majority of the metallic shaft.

11. The fastener of claim 10, wherein a proximal end of the metallic shaft and a distal end of the metallic shaft define respective parts of an outer surface of the fastener and a remaining portion of the metallic shaft therebetween is enclosed by the outer layer.

12. The fastener of claim 11, wherein the fastener is a bone screw and the proximal end of the metallic shaft is a head of the bone screw.

13. The fastener of claim 12, wherein the distal end of the metallic shaft is a distal tip of the bone screw.

14. The fastener of claim 10, wherein the outer layer is a ceramic or polyether ether ketone (PEEK) material.

15. The fastener of claim 10, wherein the outer layer is a coating and has a minimum thickness in a range from about 0.006 inches to about 0.030 inches.

16. The fastener of claim 10, wherein the outer layer is a cannulated sleeve that is removably attachable to the metallic shaft.

17. The fastener of claim 16, wherein the cannulated sleeve is open at a first end of the sleeve and at a second end of the sleeve opposite the first end such that a tip of the metallic shaft is exposed through one of the first and second ends of the sleeve.

18. The fastener of claim 16, wherein the metallic shaft and the cannulated sleeve both include a plurality of flat surfaces meeting at sharp edges such that when the cannulated sleeve is disposed over the metallic shaft, the cannulated sleeve is prevented from rotating relative to the metallic shaft.

19. The fastener of claim 10, wherein the outer layer is further configured to limit the effect of an electromagnetic field on the metallic shaft.

20. A method of using an implant in a surgical procedure comprising:
anchoring a bone screw in a bone of a patient or in a prosthesis disposed in the patient, the bone screw including a metallic shaft and a ceramic or polyether ether ketone (PEEK) outer layer enclosing an outer surface of the metallic shaft; and
conducting a magnetic resonance imaging (MRI) scan of the patient, wherein the ceramic or PEEK coating reduces temperature changes in the metallic shaft due to the scan that would occur without the ceramic or PEEK coating.
